(19) United States
(12) Patent Application Publication
(10) Pub. No.: US 2007/0009938 A1
(43) Pub. Date: Jan. 11, 2007

(54) Minimalist bZIP derivatives that bind to noncanonical gene regulatory sequences

(76) Inventor: Jumi Shin, Mississauga (CA)

Correspondence Address: BERESKIN AND PARR, 40 KING STREET WEST, BOX 401, TORONTO, ON, M5H 3Y2, CA

(21) Appl. No.: 11/442,310

(22) Filed: May 30, 2006

Related U.S. Application Data
(60) Provisional application No. 60/684,956, filed on May 27, 2005.

(51) Int. Cl. C12Q 1/68; A61K 48/00

(52) U.S. Cl. 435/6; 514/44

(57) ABSTRACT

The invention provides a method of specifically targeting noncanonical gene regulatory sequences using a minimalist basic region-leucine zipper bZIP derivative. Also provided are methods of modulating transcription and treating diseases and cancers using the minimalist basic region-leucine zipper bZIP derivatives.

This application claims the benefit under 35 USC §119(e) of U.S. provisional application No.60/684,956 filed May 27, 2005.

### FIELD OF THE INVENTION

The invention relates to the use of minimalist derivatives of the basic region/leucine zipper (bZIP) proteins that bind noncanonical DNA sites with specificity and affinity. In particular, the invention relates to the use of these derivatives for the treatment of disease and cancer.

### BACKGROUND OF THE INVENTION

The basic region/leucine zipper (bZIP) family of transcription factors comprises the simplest motif that nature uses for targeting specific DNA sites: a pair of short α-helices that recognize the DNA major groove with sequence-specificity and high affinity (Struhl, K., Ann. Rev. Biochem., 1989, 58,1051; Landschulz, W. H., et al., Science, 1988, 240, 1759-1764). Crystal structures of the bZIP domain of GCN4 bound to two different DNA sites, the AP-1 site (5′-TGACTCA) (Ellenberger, T. E., et al., Cell, 1992, 71, 1223-1237) and the CRE site (König, P. and Richmond, T. J., J. Mol. Biol., 1993, 233,139-154; Keller, W., et al., J. Mol. Biol., 1995, 254, 657-667) and the crystal structure of the Jun-Fos bZIP heterodimer bound to AP-1 (Glover, J. N. M. and Harrison, S. C., Nature, 1995, 373, 257-261) show that a continuous α-helix of ˜60 amino acids provides the basic-region interface for binding to specific DNA sites, as well as the leucine zipper coiled-coil dimerization structure. Remarkably, these crystal structures also demonstrate astonishing conservation of protein backbone structure between the two yeast GCN4 and avian Jun-Fos structures. Therefore, the simplicity and tractibility of the α-helical bZIP make it an ideal minimalist scaffold for study and design.

The protein α-helix structure is used ubiquitously for sequence-specific DNA recognition, and is one that chemists have successfully used in design and synthesis studies for many years (examples include references Dawson, P. E. and Kent, S. B. H., J. Am. Chem. Soc., 1993, 115, 7263-7266; Grove, A., et al., J. Am. Chem. Soc., 1993, 115, 5919-5924; Marqusee, S. and Baldwin, R. L., Proc. Natl. Acad. Sci. USA, 1987, 84, 8898-8902; Sasaki, T. and Kaiser, E., J. Am. Chem. Soc., 1989, 111, 380-381; Wharton, R. P. and Ptashne, M., Nature, 1985, 316, 601-605; Ghadiri, M. R. et al., J. Am. Chem. Soc., 1992, 114, 4000-4002). Proteins were previously generated with α-helical structure and DNA-recognition capabilities from a core scaffold based on the GCN4 bZIP (Lajmi, A. R. et al., J. Am. Chem. Soc., 2000, 122, 5638-5639; Lajmi, A. R., et al., Prot. Exp. Purif., 2000, 18, 394403). GCN4 is a dimeric transcriptional regulator that governs histidine biosynthesis in yeast under conditions of amino-acid starvation (Hill, D. E., et al., Science, 1986, 234, 451457). The full-length GCN4 monomer is 281 amino acids, and the bZIP comprises a dimer of ˜60-residue monomers.

Interestingly, the bZIP basic region is largely disordered until binding to DNA: both NMR and circular dichroism (CD) demonstrate that while the leucine zipper is intrinsically stable and helical, the basic region remains only loosely helical until binding to DNA (O'Neil, K. T., et al., Biochemistry, 1991, 30, 9030-9034; Saudek, V., et al., Biochemistry, 1991, 30, 1310-1317; Weiss, M. A., et al., Nature, 1990, 347, 575-578; Shin, J. A., Bioorg. Med. Chem. Lett., 1997, 7, 2367-2372; Hollenbeck, J. J. and Oakley, M. G., Biochemistry, 2000, 39, 6380-6389). This folding transition may enhance control of gene transcription. Thus, the basic region of bZIP proteins requires DNA binding to achieve stability and helicity, and this energetic requirement was circumvented by designing preorganized alanine-based scaffolds. Of the naturally occurring amino acids, alanine possesses the highest propensity for forming and stabilizing α-helical protein structures (O'Neil, K. T. and DeGrado, W. F., Science, 1990, 250, 646-651; Luque, I., et al., Biochemistry, 1996, 35,13681-13688).

Therefore, alanines were substituted into the basic regions of bacterially expressed GCN4 bZlP derivatives comprising GCN4 basic region residues 226-252 and fused to the leucine zipper from C/EBP, residues 310-338 (FIG. 1) (Lajmi, A. R., et al., Prot. Exp. Purif., 2000, 18, 394-403). The wt bZIP (wild-type) is the “native” variant comprising the GCN4 basic region and C/EBP zipper. These Ala-based mutants are short (˜100 amino acids) and hydrophobic (Ala-mutated basic regions, leucine zipper domains).

The GCN4 bZIP-DNA crystal structures show that only four highly conserved amino acids (Johnson, P. F. Mol. Cell Biol., 1993, 13, 6919-6930) in each basic region monomer make direct contacts to bases in the DNA major groove: Asn²³⁵, Ala²³⁸, Ala²³⁹, and Arg²⁴³ (Ellenberger, T. E., et al., Cell, 1992, 71, 1223-1237; König, P. and Richmond, T. J., J. Mol. Biol., 1993, 233 , 139-154; Keller, W., et al., J. Mol. Biol., 1995, 254, 657-667). 4A and 11A contain four and eleven Ala substitutions, respectively; both specific interactions with DNA bases and nonspecific Coulombic interactions with the phosphodiester backbone are maintained (Ellenberger, T. E., et al., Cell, 1992, 71, 1223-1237; König, P. and Richmond, T. J., J. Mol. Biol., 1993, 233, 139-154; Keller, W., et al., J. Mol. Biol., 1995, 254, 657-667). Additionally, 11A is mutated in the hinge region, which is important for spacing basic region monomers properly on the DNA site and can affect DNA-binding function (Sera, T. and Schultz, P. G., Proc. Natl. Acad. Sci. USA, 1996, 93, 2920-2925). The basic region mutant with the highest Ala content, 18A, retains only these four amino acids from native GCN4, plus Lys²⁴⁶ due to concerns about solubility of hydrophobic proteins (König, P. and Richmond, T. J., J. Mol. Biol., 1993, 233, 139-154). Therefore, of the 27 residues in 18A's basic region, 24 are alanine.

These Ala-rich mutants were previously found to have increasing helical structure with increasing Ala content, as well as high-affinity, sequence-specific DNA binding function. All four proteins bound to the AP-1 and CRE sites, as shown by DNase I footprinting (Lajmi, A. R. et al., J. Am. Chem. Soc., 2000, 122, 5638-5639). All four proteins displayed similar nanomolar binding affinities to both the AP-1 and CRE sites; binding to the nonspecific control duplex was >1000-fold weaker (Bird, G. H. et al., Biopolymers, 2002, 65, 10-20).

The basic-region/helix-loop-helix (bHLH) and basic-region/helix-loop-helix/leucine-zipper (bHLHZ) motifs are very similar to the bZIP in that a dimer of α-helices binds specific sites in the DNA major groove; protein dimerization is effected by the helix-loop-helix, a tetramer of α-helices in the bHLH, or by the helix-loop-helix/leucine-zipper, in which dimerization is mediated by both the tetrameric HLH and adjacent leucine zipper. Within the bHLH, there are subfamily variants: the bHLHZ, wherein a leucine zipper contiguous to Helix 2 is part of the dimerization domain (such as Max and USF), and the bHLH/PAS (such as AhR and Arnt), where the PAS domain assists in efficient protein dimerization. The PAS has been found in the Per, Arnt, and Sim proteins—hence, “PAS”—as well as AhR and HIF-1α (Gradin, K., et al., Mol. Cell. Biol. 1996, 16, 5221-5231). The PAS domain comprises 200-300 amino acids and contains characteristic repeats termed the “A” and “B” domains.

Like bZIP proteins, the bHLH/bHLHZ protein family also regulates transcription. In particular, the Myc, Max, and Mad transcription factor network comprises widely expressed bHLHZ proteins critical for control of normal cell proliferation and differentiation (Amati, B. and Land, H., Curr. Opin. Gene. Dev. 1994, 4, 102-108; Orian, A. et al., Genes Dev., 2003, 17, 1101-1114). Myc is proto-oncogenic; deregulated overexpression of myc genes leads to malignant transformation, and myc genes are suspected of being among the most frequently affected in human tumors and disease (Nesbit, C. D. et al., Oncogene 1999, 18, 3004-3016), including Burkitt lymphoma (Taub, R. et al., Proc. Natl. Acad. Sci. USA 1982, 79, 7837-7841; Dalla-Favera, R. et al. M., Proc. Natl. Acad. Sci. USA 1982, 79, 7824-7827), neuroblastomas (Schwab, M. et al., Nature 1984, 308, 288-291), and small cell lung cancers (Nau, M. M. et al., Nature 1985, 318, 69-73).

In contrast, Max is a stable, constitutively expressed dimerization partner that heterodimerizes with Myc, Mad, and Mxi, thereby controlling their DNA-binding and generegulatory activities (Amati, B. and Land, H., Curr. Opin. Gene. Dev. 1994, 4, 102-108; Orian, A. et al., Genes Dev., 2003, 17, 1101-1114). Myc-Max is a transcriptional activator that binds the Enhancer box (E-box) sequence 5′-CACGTG (Blackwood, E. M. et al., Science 1991, 251, 1211-1217; Blackwell, T. K. et al., Mol. Cell. Biol. 1993, 13, 5216-5224). Myc does not homodimerize in vivo or at physiological concentrations, so its activity is mediated by heterodimerization with Max. In contrast Max can homodimerize, although it preferentially heterodimerizes; Max homodimers can bind the E-box, albeit with lower affinities than that of the heterodimers (Blackwood, E. M. et al., Science 1991, 251, 1211-1217). Several promoters contain the E-box sequence 5′-CACGTG, including that for p53 tumor suppressor (Reisman, D. et al., Cell Growth Differ. 1993, 4, 57-65). Mad-Max (Amati, B. et al., Cell 1993, 72, 233-245) and the related Mxi-Max (Zervos, A. S. et al., Cell 1993, 72, 223-232) are transcriptional repressors that antagonize Myc-Max by competing for the same E-box sequence.

The Max network is highly conserved in vertebrates and mammals and ubiquitous; in Drosophila, for instance, a conservative estimate is that Max network proteins interact with approximately 2000 genes (Orian, A. et al., Genes Dev., 2003, 17, 1101-1114). The transactivation domain mediating the gene-regulatory activities of the Myc-Max heterodimer lies in the amino-terminal region of Myc; Max's role is to allow Myc to bind DNA, thereby mediating its cellular activities (Amati, B. and Land, H., Curr. Opin. Gene. Dev. 1994, 4, 102-108). Therefore, mutant proteins that interfere with Myc-Max recognition of the E-box site may also interfere with Myc's disease-promoting activities.

The AhR-Arnt system is notable for its possible role in disease pathways. The AhR, also known as the dioxin receptor, mediates signal transduction (Fisher, J. M., et al., Mol. Carcinogen. 1989, 1, 216-221) by dioxins and related polycyclic aromatic hydrocarbons, including benzo[a]yrenes found in cigarette smoke and smog, heterocyclic amines in cooked meat, and polychlorinated biphenyls (PCBs). In analogy to the glucocorticoid receptor, the latent AhR is found associated with heat-shock protein hsp90 in the cytosol (Cadepond, F. et al., J. Biol. Chem. 1991, 266, 5834-5841). Ligand binding induces nuclear translocation of the AhR (Pollenz, R. S. et al., Mol. Pharmacol. 1995, 45, 428-438), release of hsp90, and dimerization with the nuclear protein Arnt (Reyes, H. et al., Science 1992, 256, 1193-1195); this activated complex (Reyes, H. et al., Science 1992, 256, 1193-1195) then binds specific xenobiotic response elements and activates gene transcription (Wu, L. and Whitlock, J. P. Nucl. Acid. Res. 1993, 21, 119-125; Fujisawa-Sehara, A. et al., Nucl. Acid. Res. 1987, 15, 41794191). The endogenous ligand, if any, for the dioxin receptor has yet to be discovered. During evolution, plant flavones and later, certain combustion products like dioxin, appear to have appropriated the AhR for stimulating their own metabolism.

AhR and Arnt differ from most other bHLH transcription factors in that they both contain the PAS and that AhR-Arnt dimerization occurs only in the presence of ligand. The PAS domain is remote from the basic region, and importantly, it does not affect DNA binding, as it is purely necessary for dimerization and ligand binding; Poellinger and coworkers found that the minimal bHLH domains of AhR and Arnt are solely capable of recognition of XRE sites and dimerization (Pongratz, I., et al., Mol. Cell. Biol. 1998, 18, 4079-4088).

The crystal structures of bZIP and bHLH/bHLHZ, demonstrate that although they are distinct protein structural families, they share the most similarity in comparison to other families of DNA-binding proteins: in particular, the α-helix DNA recognition element is highly conserved in the two families (König, P. and Richmond, T. J., J. Mol. Biol. 1993, 233, 139-154; Ellenberger, T. E. et al., Cell 1992, 71, 1223-1237; Keller, W. et al., J. Mol. Biol. 1995, 254, 657-667; Glover, J. N. M. and Harrison, S. C., Nature 1995, 373, 257-26; Ma, P. C et al., Cell 1994, 77, 451-459; Ellenberger, T. et al., Genes Dev. 1994, 8, 970-980; Ferre-D'Amare, A. R. et al., Nature 1993, 363, 38-45; Brownlie, P. et al., Structure 1997, 5, 509-520; Ferre-D'Amare, A. R. et al., EMBO J. 1994, 13, 180-189). In contrast, there are differences in the hinge angles which govern positioning of the basic regions in the major grooves between bZIP and bHLH/bHLHZ. Additionally, the dimerization element in the bHLH/bHLHZ is more complicated than the smaller, simpler leucine zipper.

No simple code exists for protein-DNA recognition, and this fact has made design of sequence-specific DNA-binding proteins a major challenge.

### SUMMARY OF THE INVENTION

The present invention provides the use of minimalist bZIP derivatives for targeting noncanonical gene regulatory sequences. Accordingly, in one embodiment, the invention provides a use of a minimalist basic region-leucine zipper bZIP derivative for specifically targeting a noncanonical gene regulatory sequence. In another embodiment, the invention provides a method of specifically targeting a noncanonical gene regulatory sequence comprising administering to a cell or animal in need thereof a minimalist basic region-leucine zipper bZIP derivative, wherein the minimalist basic region-leucine zipper bZIP derivative binds to the noncanonical gene regulatory sequence. In one aspect, the noncanonical sequence may normally be recognized by Max, Myc, Arnt or HIF. In another aspect, the basic region contains alanine substitutions to create an alanine-rich mutant of the bZIP derivative. The bZIP derivatives or alanine-rich mutants thereof have widespread application in gene regulation. In particular aspects, these bZIP derivatives or alanine-rich mutants thereof are used for treating disease and cancer as well as for modulating plant gene regulation.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The invention will now be described in relation to the drawings in which:

FIG. 1. (Top) shows a schematic of expressed protein. bZIP proteins were cloned into expression vector pTrcHis B (Invitrogen), which contains a six-histidine tag for protein purification. The bZIP is at the carboxyl ternini of the expressed proteins, which is the same positioning of the bZIP domain in native GCN4. The bZIP domains comprise the basic region mutants of GCN4 (residues 226-254), leucine zipper from C/EBP (residues 310-338) plus approximately 35 residues from.the pTrcHis B expression vector (Lajmi, A. R. et al., J. Am. Chem. Soc., 2000, 122, 5638-5639; Lajmi, A. R., et al., Prot. Exp. Purif, 2000, 18, 394-403).

FIG. 1 (Bottom) shows sequences of the bZIP domains. Sequence of the bZIP domain of the wild-type protein comprises the GCN4 basic region, C/EBP leucine.zipper, plus a linker for chemical derivatization. The sequences for alanine mutants 4A, 11A, and 18A are shown below wt bZIP; these proteins are the same as wt bZIP, except for the mutated basic regions. Alanine substitutions are underlined and highly conserved bZIP residues are in bold. We note that amino acid 227 is arginine in both 4A and 11A; this is a cloning artifact, and this residue has no interaction with DNA (Ellenberger, T. E., et al., Cell, 1992, 71, 1223-1237; König, P. and Richmond, T. J., J. Mol. Biol., 1993, 233,139-154; Keller, W., et al., J. Mol. Biol., 1995, 254, 657-667).

FIG. 2. (A) shows sequences of all the DNA sites used in DNase I footprinting experiments. All sequences are duplexes. Core target sequences are in bold. The Partial site actually contains a weak half site, 5′-AGAC-3′, which is italicized, and the full Partial site is in bold. The flanking sequences surrounding the AP-1 and XRE1 target sites are from the his3 and CYP1A1 promoter regions, respectively. Flanking sequences surrounding C/EBP, HRE, and the Partial site are the same as those for EMSA.

FIG. 2 (B) shows sequences of DNA sites used in EMSA experiments. All sequences are 24-mer duplexes for full sites and 20-mer duplexes for half sites. The core target sequences are in bold; the inserts between flanking sequences are underlined. The flanking sequences were chosen to minimize DNA secondary structure. The flanking sequences are identical for all duplexes, except that the flanking sequences of WT AP-1 are from the his3 gene from yeast. Note that for the Partial site, the actual target is shifted by one base pair. The NS sequence is a nonspecific DNA control. The two thymines at the 3-end of each duplex were P³²-labeled.

FIG. 3 shows DNase I footprinting analysis on expressed wt bZIP bound to AP-1, C/EBP, XRE1, E-box, HRE, and Partial site. Data presented for 5′ ³²P-endlabeled DNA (˜20,000 cpm/lane). Lanes 1, 5, 9, 13, 17, and 21: chemical sequencing G reactions. Lanes 2, 6, 10, 14, 18, and 22: DNase I cleavage control reactions. Lanes 3, 7, 11, and 23: DNase I cleavage reactions with 0.5 μM wt bZIP. Lanes 15 and 19: DNase I cleavage reactions with 1 μM wt bZIP. Lanes 4, 8, 12, 16, 20, and 24: DNase I cleavage reactions with 3 μM wt bZIP. The positions of the corresponding DNA targets are indicated. The results were reproduced at least three times,

FIG. 4 shows EMSA on synthetic wt bZIP bound to all full and half sites. Each lane contains ˜3000 cpm ³²P-endlabeled DNA 24mer duplex (full sites) or 20-mer duplex (half sites). EMSA was run at 120 V for 90 min. I indicates free DNA; II indicates bandshift from dimeric wt bZIP complexation. FIGS. A and B represent different EMSA gels. A) EMSA on synthetic wt bZIP bound to full sites. Lane 1: free AP-1 DNA. Lanes 2-11: DNA in the presence of 1500 nM synthetic wt bZIP. DNA target sites used in Lanes 2-11 are wr AP-1, AP-1, CRE, C/EBP, XRE1, Arnt E-box, Max E-box, HRE, Partial site, and nonspecific control, respectively. B) EMSA on synthetic wt bZIP bound to half sites. Lane 1: free AP-1 half site DNA. Lanes 2-5: DNA in the presence of 1500 nM synthetic wt bZIP. DNA target sites used in Lanes 2-5 are the half sites of AP-1, C/EBP, Arnt E-box, and Max E-box, respectively.

FIG. 5 shows another representative EMSA. Five EMSA gels showing wt bZIP binding to AP-1, XRE1, C/EBP, Arnt E-box (E-box preferred by Arnt), and Max E-box (E-box preferred by Max). Each gel is labeled, and DNA sequence is shown. Above each gel is listed concentrations of protein monomer; from left to right, 0 micromolar wt bZIP to 10 micromolar wt bZIP. Lower band is unbound DNA; as concentration of wt bZIP increases, protein then binds DNA and the DNA shifts up (upper band). Many EMSA experiments were performed on controls, 4A, 11A, and 18A. The gels all look similar.

FIG. 6 shows data work-up of a representative gel (such as those shown in FIG. 5) used to compute dissociation constants. The value “m1” shows the dissociation constant. For example, wt bZIP binds to AP-1 (red curve) with a dissociation constant of 568 nanomolar. Error is shown in the right column. “Chisq” and “R” show the goodness of fit for each curve.

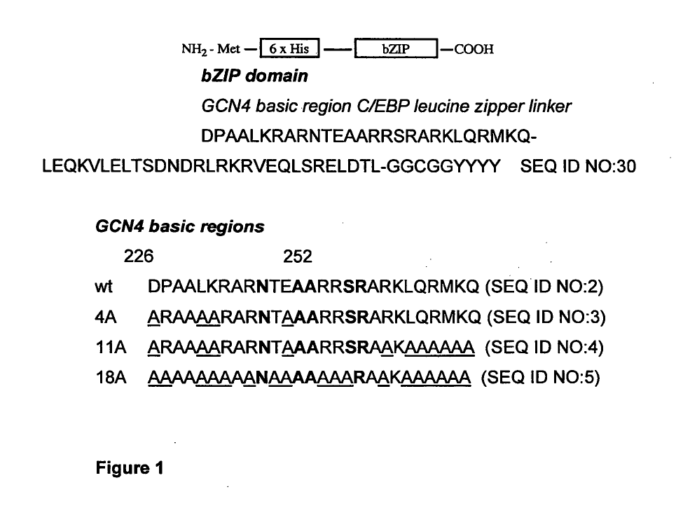

### DETAILED DESCRIPTION OF THE INVENTION

The present inventor previously generated the wt bZIP, which is a GCN4 derivative (GCN4 basic region+C/EBP leucine zipper) and also generated 4A, 11A, and 18A alanine-rich derivatives of wt bZIP; that is, simplified versions of wt bZIP. Therefore, all four proteins are derivatives of GCN4. GCN4 binds to the AP-1 site (5′-TGACTCA) (SEQ ID NO:1), and that is GCN4's canonical binding site. It was previously shown that the basic regions of wt Bzip (SEQ ID NO:2), 4A (SEQ ID NO:3), 11A (SEQ ID NO:4), and 18A (SEQ ID NO:5) all bind the canonical AP-1 site (as well as the related, canonical ATF/CREB site, also called the CRE site 5′-TGACGTCA (SEQ ID NO:6)); these were shown to footprint (Lajmi, A. R. et al., J. Am. Chem. Soc., 2000, 122, 5638-5639) and binding measurements showed that all four derivatives bound AP-1 and ATF/CREB with almost identical dissociation constants (Bird, G. H. et al., Biopolymers, 2002, 65, 10-20). Therefore, all four derivatives are approximately equally good at sequence-specific, high-affinity DNA-binding function. Almost all of the native amino acids can be replaced in the GCN4 basic region with alanine, and still retain native DNA-binding function.

In the present invention, it is demonstrated that the WT bZIP and alanine mutants also bind noncanonical DNA sites with specificity and affinity and can specifically target gene regulatory sequences: C/EBP (CCMT/enhancer binding protein, 5′-TTGCGCAA (SEQ ID NO:7)), XRE1 (xenobiotic response element, 5′-TTGCGTGA(SEQ ID NO:8)), HRE (HIF response element, 5′-GCACGTAG (SEQ ID NO:9)), and two E-box derivatives (5′-TCACGTGA (SEQ ID NO:10), preferred site for Max and Myc; 5′-CCACGTGG (SEQ ID NO:11), preferred site of Arnt). These GCN4 derivatives still target the AP-1 site with strongest affinity, followed by slightly weaker binding to the C/EBP site, then weaker binding to XRE1 and HRE, and very weak, yet specific, binding to both E-box sites. The present inventors have also demonstrated that binding to nonspecific control DNA sites is much weaker, over ten-fold weaker than binding to the E-box sites.

As such, these proteins can be used to fine-tune binding to. noncanonical sites; that is, to generate a slightly weaker binder, or a binder with a certain dissociation constant. The Ala-rich mutants may be preferred embodiments that will do the same thing as wt bZIP (depends on the purpose); the Ala-rich mutants are simple, but they are more hydrophobic and uncharged, so they have a tendency to precipitate out of aqueous solutions. However, in an organic solution, a fatty micelle or fatty tissue, the Ala-rich mutants may be preferred.

Accordingly, the present invention provides the use of basic region-leucine zipper minimalist bZIP derivatives for specifically targeting a noncanonical gene regulatory sequence. In another embodiment, the invention provides a method of specifically targeting a noncanonical gene regulatory sequence comprising administering to a cell or animal in need thereof a basic region-leucine zipper minimalist bZIP derivative, wherein the basic region-leucine zipper minimalist bZIP derivative binds the noncanonical gene regulatory sequence. For example, the method may comprise adding a basic region-leucine zipper minimalist bZIP derivative to a cell or sample that has a gene regulated by a noncanonical gene regulatory sequence, allowing the basic region-leucine zipper minimalist bZIP derivative to target the noncanonical gene regulatory sequence. In a particular aspect, targeting the noncanonical gene regulatory sequence results in modulation of transcription of the gene.

The term “cell” as used herein includes all types of cell.s The cell is preferably plant or animal. The term “animal” as used herein includes all members of the animal kingdom including humans. The animal is preferably human.

The term “basic region-leucine zipper minimalist bZIP derivative” as used herein means-the basic region of a bZIP protein fused to the leucine zipper domain of either the same or a different bZIP protein. In one aspect, the basic region and/or leucine zipper is derived from a bZIP protein selected from the group consisting of Fos, Jun, GCN4 (general control of nitrogen and purine metabolism factor-4), VBP (vitellogenin gene binding protein), GBF-1 (G-box factor-1), opaque, DBP (D-box binding protein), CHOP-10 (C/EBP homologous protein-10), CREB (CRE binding protein), C/EBP (CCAAT/enhancer binding protein), PAR (proline- and acidic amino acid-rich protein) and ATF2 (activating transcription factor-2). In a particular aspect, the basic region is derived from GCN4 and the leucine zipper region is derived from C/EBP.

The term “specifically targets a noncanonical sequence” means binds to a noncanonical sequence with at least some measurable binding affinity and targeting specificity, but that is not the canonical sequence, which is the sequence targeted natively/in vivo. Realistically, binding at a noncanonical site, although targeted with sequence specificity, will be weaker (lower binding affinity) than binding to the canonical site. However, in the case of binding to CIEBP site, only 10-fold reduced binding affinity, and binding to XRE1 is 20-fold reduced. Thus, the bZIP derivatives are s to the noncanonical sites. In this system, the degree of binding affinity can be modulated to a desired level. For example, it may be useful to have a bZIP derivative that comes off and on, allowing regulation of transcription downstream.

The term “noncanonical gene regulatory sequence” as used herein means the sequence is not the usual DNA binding site for the bZIP protein from which the basic region is derived. Noncanonical is NOT the same as nonspecific DNA. In one aspect, the noncanonical gene regulatory sequence is selected from the group consisting of C/EBP (CCMT/enhancer binding protein, 5′-TTGCGCAA (SEQ ID NO:7)), XRE1 (xenobiotic response element, 5′-TTGCGTGA (SEQ ID NO:8)), HRE (HIF response element, 5′-GCACGTAG (SEQ ID NO:9), and two E-box derivatives (5′-TCACGTGA (SEQ ID NO:10), preferred site for Max and Myc; 5′-CCACGTGG (SEQ ID NO:11), preferred site of Arnt). In a particular aspect, the bZIP derivatives specifically target the noncanonical gene regulatory sequence that is normally recognized by Max. In another particular aspect, the bZIP derivatives specifically target the noncanonical gene regulatory sequence that is normally recognized by Myc. In a further particular aspect, the bZIP derivatives specifically target the noncanonical gene regulatory sequence that is normally recognized by the AhR-Arnt complex. In yet another aspect, the bZIP derivatives specifically target the noncanonical gene regulatory sequence that is normally recognized by C/EBP. In yet another aspect, the bZIP derivatives specifically target the noncanonical gene regulatory sequence that is normally recognized by HIF.

In a further embodiment, the basic region derived from a bZIP protein has alanine substitutions. In a particular aspect, the basic region is derived from GCN4 and has 4, 11 or 18 alanine substitutions.

Thus, both DNA-binding specificity and affinity are maintained with all the bZIP derivatives binding to noncanonical gene regulatory sequences. The bZIP scaffold may be a powerful tool in design of small, α-helical proteins with desired DNA-recognition properties capable of serving as therapeutics targeting transcription.

Myc proteins are involved in disease, tumors, cancers, stroke and heart attacks. The bZIP derivatives of the invention that bind the E-box may interfere with Myc's disease promoting activities. Accordingly, the invention provides the use of the minimalist bZIP derivatives that target the E-box DNA for repressing myc-related transcriptional activation. The invention also provides a method of repressing myc-related transcriptional activation comprising administering to a cell or animal in need thereof the minimalist bZIP derivatives that target the E-box DNA. In a particular embodiment, the method comprises administering the minimal bZIP derivative that targets the E-box DNA to an animal cell, preferably a human cell.

Myc is also an oncoprotein known to be overexpressed in a wide variety of human cancers, including 80% breast, 70% colon, and 90% gynecological cancers, 50% hepatocellular carcinomas and a variety of hematological tumors. Accordingly, in another embodiment, the invention provides the use of the minimalist bZIP derivatives that target the E-box DNA for treating cancer. The invention also provides a method of treating cancer comprising administering to a cell or animal in need thereof a minimalist bZIP derivative that targets the E-box DNA. In a preferred embodiment, the animal is human. In another preferred embodiment, the cancer is selected from the group consisting of Burkitt lymphoma, neuroblastoma, small cell lung cancer, breast cancer, colon cancer, liver cancer, gynecological cancer, hepatocellular carcinomas and hematological tumors, bladder cancer, gastric cancer, melanoma, myeloma, ovarian cancer, prostate cancer.

HIF gets turned on under conditions of hypoxia (low oxygen), and it turns on a number of genes involved in angiogenesis and glycolysis, which are important for disease cells to grow. The bZIP derivatives that bind the HRE sequence may interfere with HIF's disease promoting activities. Accordingly, the invention also provides the use of minimalist bZIP derivatives that target the HRE sequence for repressing HIF-related transcriptional activation. The invention also provides a method of repressing HIF-related transcriptional activation comprising admininstering to a cell or animal in need thereof a minimalist bZIP derivative that targets the HRE sequence, wherein the minimalist bZIP derivative binds the HRE sequence. In a particular embodiment, the method comprises administering the minimalist bZIP derivative that targets the HRE sequence to an animal cell, preferably a human cell.

The minimalist bZIP derivatives may also be fused to a repressor of transcription. Accordingly, the invention provides the use of a minimalist bZIP derivative that targets a noncanonical gene regulatory sequence fused to a repressor for repressing transcriptional activation. The invention also provides a method of repressing transcriptional activation comprising administering to a cell or animal in need thereof a minimalist bZIP derivative that targets a noncanonical gene regulatory sequence fused to a repressor. In one embodiment, the noncanonical gene regulatory sequence is E-box and the transcriptional activation is related to Myc. In another embodiment the noncanonical gene regulatory sequence is HRE and the transcriptional activation is related to HIF.

In another embodiment, the bZIP derivative fused to a repressor targets an E-box DNA sequence for treating cancer. The invention also provides a method of treating cancer comprising administering to a cell or animal in need thereof a bZIP derivative that targets an E-box DNA sequence fused to a repressor. In a preferred embodiment, the animal is human. In another preferred embodiment, the cancer is selected from the group consisting of Burkitt lymphoma, neuroblastoma small cell lung cancer, breast cancer, colon cancer, liver cancer, gynecological cancer, hepatocellular carcinomas and hematological tumors, bladder cancer, gastric cancer, melanoma, myeloma, ovarian cancer, prostate cancer.

The Myc, Max and Mad transcription factor network are critical for control of normal cell proliferation and differentiation. Accordingly, the invention also provides the use of the minimalist bZIP derivatives of the invention that target the E-box DNA for controlling cell proliferation and/or differentiation. The invention also provides a method of controlling cell proliferation and/or differentiation comprising administering to a cell or animal in need thereof a minimalist bZIP derivative that targets the E-box DNA. In another embodiment, the invention provides the use of a minimalist bZIP derivative that targets the E-box DNA fused to an activation domain for activating cell proliferation and/or differentiation. The invention also provides a method of activating cell proliferation and/or differentiation comprising administering to a cell or animal in need thereof a minimalist bZIP derivative that targets the E-box DNA fused to an activation domain. In yet another embodiment, the invention provides the use of a minimalist bZIP derivative that targets the E-box DNA fused to a repressor for repressing cell proliferation and/or differentiation. The invention also provides a method of repressing cell proliferation and/or differentiation comprising administering to a cell or animal in need thereof a minimalist bZIP derivative that targets the E-box DNA fused to a repressor.

The Ahr/Arnt system is notable for its possible role in disease pathways given its role in mediating signal transduction by dioxins and related polycyclic aromatic hydrocarbons. 2,3,7,8-tetrachlorodibenzo-p-dioxin, commonly referred to as TCDD or dioxin, produces a variety of highly toxic effects, including chloracne, teratogenesis, tumor promotion, and immunotoxicity (Whitelaw, M. et al., Mol. Cell Biol. 1993, 13, 2504-2514; Poland, A. and Knutson, J. C., Ann. Rev. Pharmacol. Toxicol. 1982, 22, 517). Dioxin is an industrial byproduct produced during herbicide manufacture, the bleaching of paper pulp, and combustion of chlorinated organic materials. Dioxin can accumulate in the environment; although it decomposes rapidly in organic solution under artificial or natural light; no photodecomposition occurs in aqueous environments or on wet or dry soil (Crosby, D. G., et al., Science, 1971, 173, 748-749). The resistance of dioxin to metabolic degradation and the stability of the dioxin-receptor complex may account for its persistence and toxicity (Johnson, E. F., Science, 1991, 252, 924).

Animal studies have proven this ubiquitous pollutant to be extremely lethal, perhaps the most powerful carcinogen tested (Roberts, L., Science, 1991, 251, 624-626; Gray, L. E. Jr. and Ostby, J. S., Toxicol. Appl. Pharmacol., 1995, 133, 285-294). Human effects, however, have been subject to wide controversy, especially in studies concerning dioxin-tainted Agent Orange used by the United States during the Vietnam War as a defoliant.

A study examined the effects of a chemical plant explosion that occurred twenty years ago in Seveso, Italy (Bertazzi, P. A., et al., Epidemiology, 1993, 4, 398-406). The 36,000 people exposed to dioxin were compared with 180,000 people living nearby in uncontaminated areas. No conclusions could be drawn about the high exposure group because it contained too few people. In the moderate exposure group, liver cancer was three times higher than in the control population; men showed increased cancer of the lymph nodes, and women displayed elevated incidence of multiple myeloma and leukemia. The low exposure group had increased incidences of soft tissue sarcoma and non-Hodgkin's lymphoma.

In 1991, the National Institute of Occupational Safety and Health published an exhaustive examination of the mortality records of 5172 male chemical workers exposed to dioxin on the job from 1942 to 1982 (Fingerhut, M. A., et al., N. Engl. J. Med., 1991, 324, 212-218). The low exposure cohort worked for less than one year in a dioxin-tainted occupation, the high exposure cohort for at least a year; the latency period for cancer occurrence was at least twenty years, meaning that these men were first exposed to dioxin at least twenty years earlier (Roberts, L., Science, 1991, 251, 624-626). The low-exposure group showed no increased risk in cancer, despite exposure to dioxin at levels 90 times higher than that for the general population. The high exposure group, however, was estimated to be exposed to dioxin levels 500 times higher than that for the general population and had nearly a 50% increase in cancer mortality, mostly in soft tissue sarcomas and, unexpectedly, respiratory cancer.

Despite dioxin's powerful cancer-inducing properties, dioxin has been shown to exhibit antiestrogenic activity which works through the AhR (Rowlands, C., et al., Cancer Res., 1993, 53, 1802-1807). TCDD, in specific,. inhibits estrogen-induced proliferation in human ovarian carcinoma cell lines. TCDD is, however, highly toxic and unacceptable for clinical trials, but other nontoxic alkylated polychlorinated dibenzofurans that bind the AhR and exhibit antiestrogenic activity against estrogen-dependent ovarian and mammary tumors are being examined (Rowlands, C., et al., Cancer Res., 1993, 53, 1802-1807).

Given that the endogenous ligand for the dioxin receptor is not yet known, the minimalist bZIP derivatives of the invention that target the XRE1 site are useful for regulating the dioxin pathway. Accordingly, the invention also provides the use of the minimalist bZIP derivatives that target the XRE1 site for modulating the dioxin pathway. The invention also provides a method of modulating the dioxin pathway comprising administering to a cell or animal in need thereof a minimalist bZIP derivative that targets the XRE1 site, In another embodiment, the invention provides the use of a minimalist bZIP derivative that targets the XRE1 site fused to an activation domain for activating the dioxin pathway. The invention also provides a method of activating the dioxin pathway comprising administering to a cell or animal in need thereof a minimalist bZIP derivative that targets the XRE1 site fused to an activation domain. Transcriptional activation at XRE1 may be effective against estrogen-dependent diseases, including some forms of estrogen-dependent ovarian and mammary cancers and tumors. In yet another embodiment, the invention provides the use of a minimalist bZIP derivative that targets the XRE1 site fused to a repressor domain for repressing the dioxin pathway. The invention also provides a method of repressing the dioxin pathway comprising administering to a cell or animal in need thereof a minimalist bZIP derivative that targets the XRE1 site fused to a repressor. In yet a further embodiment, the invention provides the use of the minimalist bZIP derivatives that target the XRE1 site for treating cancer. The invention also provides a method of treating cancer comprising administering to a cell or animal in need thereof a minimalist bZIP derivative that targets the XRE1 site. In a preferred embodiment, the animal is human. The cancer may be selected from the group consisting of liver cancer, cancer of the lymph nodes, multiple myeloma, leukemia, soft tissue sarcoma, non-Hodgkin's lymphoma, respiratory cancer, which are all affected by dioxin.

Since the minimalist bZIP derivatives of the invention bind to specific DNA sequences, the proteins may be used as targeting agents. In another embodiment, the minimalist bZIP derivatives are fused to a drug. For example, the drug may be an anti-cancer agent.

The invention also provides a pharmaceutical composition for targeting noncanonical gene regulatory sequences comprising a minimalist bZIP derivative and a pharmaceutically acceptable carrier, diluent or excipient. The noncanonical gene regulatory sequences may be selected from the group consisting of E-box (SEQ ID NOs:10 and 11), XRE1 (SEQ ID NO:8), C/EBP (SEQ ID NO:7) and HRE for all sites are targeted with sequence-specificity.

In a particular embodiment, the invention provides a pharmaceutical composition for treating a mammal with cancer comprising a minimalist bZIP derivative that targets the E-box and a pharmaceutically acceptable carrier, diluent or excipient. In another embodiment, the invention provides a pharmaceutical composition for treating a mammal with cancer comprising a minimalist bZIP derivative that targets the E-box fused to a repressor and a pharmaceutically acceptable carrier, diluent or excipient.

In one aspect, the cancer is selected from the group consisting of Burkitt lymphoma, neuroblastoma, small cell lung cancer, breast cancer, colon cancer, liver cancer, gynecological cancer, hepatocellular carcinomas and hematological tumors, bladder cancer, gastric cancer, melanoma, myeloma, ovarian cancer, prostate cancer, which are affected by Myc transcriptional activation.

In one embodiment, the invention provides a pharmaceutical composition for treating a mammal with cancer comprising a minimalist bZIP derivatve that targets the XRE1 sequence and a pharmaceutically acceptable carrier, diluent or excipient. In another embodiment, the invention provides a pharmaceutical composition for treating a mammal with cancer comprising a minimalist bZIP derivative that targets the XRE1 sequence fused to a repressor and a pharmaceutically acceptable carrier, diluent or excipient. In one aspect, the cancer is selected from the group consisting of liver cancer, cancer of the lymph nodes, multiple myeloma, leukemia, soft tissue sarcoma, non-Hodgkin's lymphoma, respiratory cancer are all affected by dioxin. Transcriptional activation at XRE1 may also be effective against estrogen-dependent diseases, including some forms of estrogen-dependent ovarian and mammary cancers and tumors.

The bZIP derivatives may be formulated into pharmaceutical compositions for administration to subjects in a biologically compatible form suitable for administration in vivo. By “biologically compatible form suitable for administration in vivo” is meant a form of the substance to be administered in which any toxic effects are outweighed by the therapeutic effects. The substances may be administered to living organisms including humans, and animals. Administration of a therapeutically active amount of the pharmaceutical compositions of the present invention is defined as an amount effective, at dosages and for periods of time necessary to achieve the desired result. For example, a therapeutically active amount of a substance may vary according to factors such as the disease state, age, sex, and weight of the individual, and the ability of protein to elicit a desired response in the individual. Dosage regime may be adjusted to provide the optimum therapeutic response. For example, several divided doses may be administered daily or the dose may be proportionally reduced as indicated by the exigencies of the therapeutic situation.

The active substance may be administered in a convenient manner such as by injection (subcutaneous, intravenous, intramuscular, etc.), oral administration, inhalation, transdermal administration (such as topical cream or ointment, etc.), or suppository applications. Depending on the route of administration, the active substance may be coated in a material to protect the compound from the action of enzymes, acids and other natural conditions which may inactivate the compound.

The compositions described herein can be prepared by per se known methods for the preparation of pharmaceutically acceptable compositions which can be administered to subjects, such that an effective quantity of the active substance is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 1985). On this basis, the compositions include, albeit not exclusively, solutions of the substances in association with one or more pharmaceutically acceptable vehicles or diluents, and contained in buffered solutions with a suitable pH and iso-osmotic with the physiological fluids.

Additionally, these minimalist bZIP derivatives have agricultural and biological (nonhuman) applications. For example, the plant G-box is 5′-CACGTG, the same sequence as the mammalian E-box; the G-box refers specifically to plants. Thus, the bZIP derivatives can also bind the G-box, as it is identical to the E-box. Plants use bZIP proteins ubiquitously; =i Arabidopsis thaliana has four times as many bZIP proteins as do humans and yeast (Jakoby, M. et al., Trends Plant Sci., 2002, 7, 106-111). In Arabidopsis, many G-box regulated genes are linked to ultraviolet and blue light signal transduction and regulation of light-sensitive promoters, and there is evidence that some GBF-like proteins (G-box binding factor), including ROM1 and ROM2, may regulate storage protein expression, and therefore, play a role in seed maturation. Control of storage protein expression may achieve healthier, more vigorous, larger plants and crops. Accordingly, the present invention provides the use of minimalist bZIP derivatives that target a G-box DNA sequence for modulating G-box regulated genes. The invention also provides a method of modulating G-box regulated genes comprising administering to a cell a minimalist bZIP derivative that targets a G-box DNA sequence, wherein the minimalist bZIP derivative binds the G-box DNA sequence. In a preferred embodiment the cell is a plant cell.

In addition, the E-box/G-box, XRE1, C/EBP, or HRE may be cloned upstream of a gene that one wishes to control: hence, producing a genetically modified plant. This plant could then be engineered to express a bZIP derivative that would then target the cloned DNA site, thereby, regulating the desired gene. By extension, the genetically modified organism does not have to be a plant, but it could be an animal; these proteins can also have veterinary applications in cases of genes that fortuitously possess an E-box (or E-box-related) or XRE1 (or XRE1-related) or C/EBP (or C/EBP-related) or HRE (or HRE-related) sequence in the promoter.

The bZIP derivatives offer a significant tool for controlling gene transcription in plants. This has many applications including activating or repressing endogenous growth proteins. The bZIP derivative can compete with native proteins for binding DNA and thus affect endogenous transcription. For example, regulation of genes important for proper grape growth in the wine industry, the ability to fight common grape afflictions, such as phylloxera, or regulation of tissue-specific growth in plants.

The bZIP derivatives can be used to control a variety of traits in plants such as size, moistness, quality, durability and texture. Both monocotyledonous and dicotyledonous plants can be used, as well as vegetables, fruit and crops.

The above disclosure generally describes the present invention. A more complete understanding can be obtained by reference to the following specific examples. These examples are described solely for the purpose of illustration and are not intended to limit the scope of the invention. Changes in form and substitution of equivalents are contemplated as circumstances might suggest or render expedient. Although specific terms have been employed herein, such terms are intended in a descriptive sense and not for purposes of limitation.

The following non-limiting examples are illustrative of the present invention:

### EXAMPLE

DNase I footprinting was used to demonstrate that the Ala-rich mutants bind specifically to the C/EBP consensus sequence (CCAAT/enhancer binding protein, 5′-TTGCGCM (SEQ ID NO:7)), which is bound by the C/EBP family of transcription factors that regulate genes involved in a variety of functions, including activation of constitutive and acute phase responsive genes in the liver, control of adipocyte differentiation, and regulation of inflammatory cytokine genes and other activities of monocytic cells (Johnson, P. F., Mol. Cell. Biol., 1993, 13, 6919-6930); the XRE1 site (xenobiotic response element 1, 5′-TTGCGTGA (SEQ ID NO:8)), which is a member of the xenobiotic response element family that lies in enhancers of target genes and when recognized by the aryl hydrocarbon receptor (AhR) and Arnt heterodimer, promotes transcription of a battery of xenobiotic-metabolizing enzymes—specifically XRE1 resides in the 5′-flanking region of the CYP1A1 (cytochrome p450) gene; the HRE site (HIF response element, 5′-GCACGTAG (SEQ ID NO:9)), which resides in enhancer regions of target genes involved in glycolysis, erythropoiesis (upregulation of erythropoietin transcription), and angiogenesis (upregulation of vascular endothelial growth factor transcription) (Lando, D., et al., Eur. J. Biochem., 2003, 270, 781-790) and is bound by the HIF-1α/Arnt heterodimer under conditions of hypoxia; and two E-box derivatives (5′-TCACGTGA (SEQ ID NO:10), preferred site for Max and Myc; 5′-CCACGTGG (SEQ ID NO:11), preferred site of Arnt), which is bound by Max heterodimerized to either Mad or Myc, an oncoprotein known to be overexpressed in a wide variety of human diseases, including 80% breast, 70% colon, and 90% gynecological cancers, 50% heptacellular carcinomas and a variety of hematological tumors (Gardner, L., et al., Encyclopedia of Cancer, 2002, Bertino, J. R. Ed., Academic Press, San Diego, Calif.).

C/EBP is a known bZIP protein, like GCN4; however, C/EBP (Vinson, C. R., et al., Science, 1989, 246, 911-916) is found in numerous species including mammals (humans, rats), vertebrates (chicken), and invertebrates (fruit flies), whereas GCN4 is native to yeast (Struhl, K., Ann. Rev. Biochem., 1989, 58, 1051). The other proteins discussed in the above paragraph belong to the basic/helix-loop-helix (bHLH) family of transcription factors. Specifically, AhR, Arnt, and HIF-1α are members of the bHLH/PAS family (Hogenesch, J. B., et al., J. Biol. Chem., 1997, 272, 8581-8593; Pongratz, I., et al., Mol. Cell. Biol. 1998, 18, 4079-4088), which comprises an adjacent secondary dimerization domain Per/Amt/Sim (PAS); these proteins are found in mammals including humans, rabbits, and rats. Max, Mad, and Myc belong to the bHLH/Z family (Nair, S. K. and Burley, S. K., Cell, 2003, 112,193-205; O'Hagan, R. C., et al., Nat Genet., 2000, 24, 113-119), whose secondary dimerization domain is a leucine zipper adjacent to the bHLH; similarly, these proteins are also found widespread in mammals, vertebrates, and invertebrates.

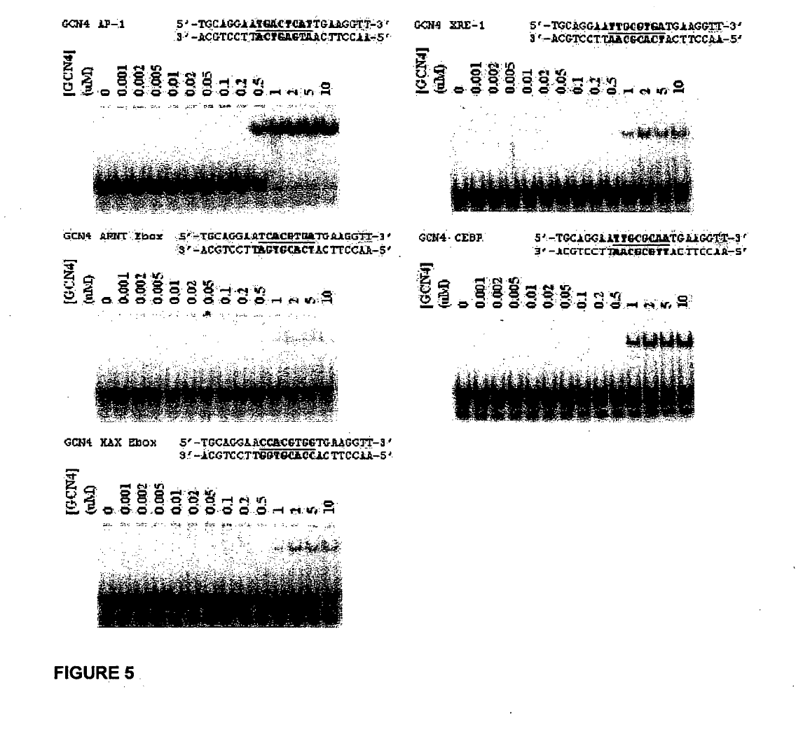

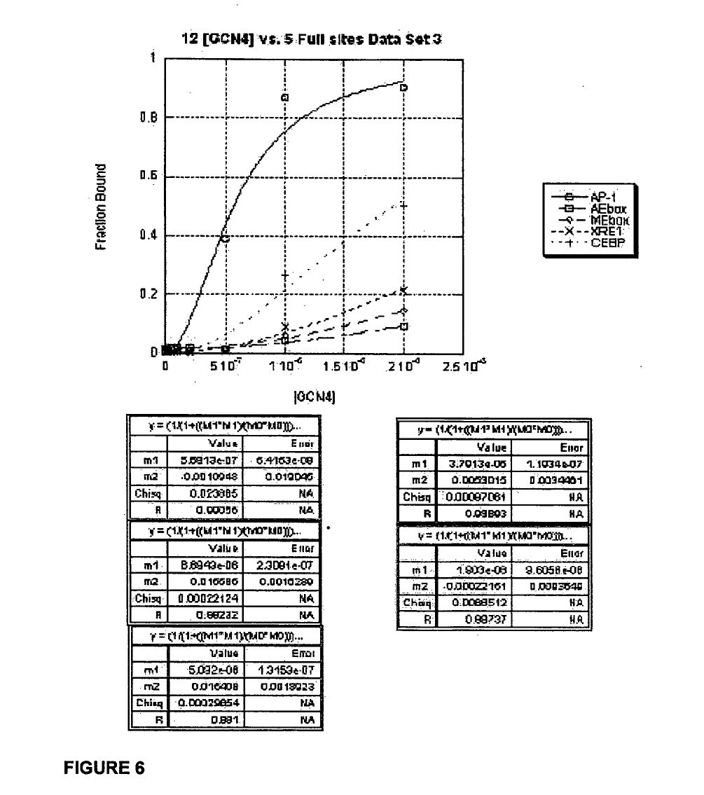

Electrophoretic mobility shift assays were used to quantify binding of the Ala-rich mutants to these sites, as well as half sites, and the effects of changing flanking sequences was examined (FIGS. 5 and 6). These GCN4 derivatives still target the AP-1 site with strongest affinity, followed by slightly weaker binding to the C/EBP site (approximately three-fold weaker binding than to AP-1), then weaker binding to XRE1 and HRE (approximately seven-fold weaker binding than to AP-1), and very weak, yet specific, binding to both E-box sites (approximately ten-fold weaker binding than to AP-1). Binding to nonspecific sequences was much weaker. The dissociation constants of the protein-DNA complexes were measured under equilibrium conditions. K_{d} values reveal protein-DNA binding in the nanomolar-micromolar range. Here we show that these highly simplified, Ala-rich, α-helical bZIP derivatives of GCN4 can also bind with high-affinity and sequence specificity to noncanonical, noncognate gene regulatory sequences. These results demonstrate that the bZIP possesses the versatility and capability to bind a variety of sequences with varying affinities; this property can be further exploited for the fine-tuning of a designed protein's binding affinity to a target site.

Electrophoretic Mobility Shift Assays were also used to quantify binding of wt bZIP to noncognate sites C/EBP, XRE1, HRE and E-box as well as derivatives of these full and half sites. The effects of changing the flanking sequences was also examined. The full-site binding affinities gradually decrease from cognate sites AP-1 and CRE with K_{d} values 13 and 12 nM, respectively, to noncognate sites (in decreasing order of binding) C/EBP, XRE1, two E-box derivatives, and HRE with K_{d} values 120 nM to low μM range. DNA-binding specificity at half sites is maintained; however, half-site binding affinities rapidly decrease from the cognate half site (K_{d} 84 nM) to noncognate half sites, all with K_{d} values>2 μM. The half-site results are in agreement with other work demonstrating that the bZIP targets DNA through the monomer-binding pathway, which enhances sequence specificity. This work demonstrates that the bZIP scaffold may be a powerful tool in design of small, α-helical proteins with desired DNA-recognition properties.

Materials and Methods

General

Chemically synthesized proteins were purchased from BioMer Technology. [α-³²P]-dTTP was supplied by Amersham Biosciences (GD Healthcare). DNA oligonucleotides used for cloning proteins were synthesized on a PE Biosystems Expedite 8909 at the DNA Synthesis Facility, University of Pittsburgh. DNA oligonucleotides used for EMSA experiments were purchased from Operon. Enzymes were obtained from New England Biolabs and used with the supplied buffers. Reagents were purchased from Aldrich, Acros, BioShop, Bio-Rad and Fisher. Water was purified with a Milli Q filtration system from Millipore.

HPLC purification was performed on a Beckman System Gold with Beckman and Vydac reverse-phase columns. Mass spectra was recorded on a Waters Micromass® ZQ™ Mass Spectrometer at the Department of Chemistry, University of Toronto at Mississauga. Radioactivity was monitored on a Beckman LS 6500 scintillation counter. UV/Vis spectra were recorded on a Beckman DU 640 Spectrophotometer. Visualization and quantification of EMSA signals were performed on a Storm 840 PhosphorImager and ImageQuant® ver. 5.2 software (Molecular Dynamics, Amersham Pharmacia Biotech) at the Department of Biology, University of Toronto at Mississauga.

Gene Assembly

Protocols for DNA oligonucleotide synthesis and gene construction have been described in detail elsewhere (Lajmi, A. R., et al., Prot. Exp. Purif., 2000, 18, 394-403). A brief summary of these procedures follows: genes for expression of bZIP proteins were constructed by mutually primed synthesis, followed by polymerase chain reaction with terminal primers for gene amplification and purification by nondenaturing polyacrylamide-gel electrophoresis. Duplex DNA was then cloned into protein expression vector pTrcHis B (Invitrogen); pTrcHis expresses proteins with a six-histidine tag for purification purposes.

Transformation and Selection for Protein Expression

Recombinant plasmids were transformed into the E coli strain TOP10® (Invitrogen) by electroporation (Bio-Rad). TOP10® was used as the maintenance-host line to reserve correct plasmids without unwanted recombination. Cloned inserts were sequenced at the Centre for Applied Genomics, Hospital for Sick Children, University of Toronto. Clones with correct-insert plasmids were stored at −80° C. in 20% glycerol (w/v).

For the purpose of protein expression, plasmids with correct inserts were freshly transformed into the E Coli strain BL21 (DE3) (Stratagene) to avoid unwanted recombination. Colonies of BL21(DE3) with recombined plasmids were screened by initial protein expression in order to determine whether the clones overexpress desired proteins. Colonies selected for initial protein expression were first grown in 3 ml of LB medium containing 100 ug/ml ampicillin for 16 hours at 37° C.; 60 ul of this overnight culture was added to another 3 ml of LB medium with 100 ug/ml ampicillin and grown at 30° C. until late-log phase (OD600˜1.4). IPTG was added to a final concentration of 1 mM, and the culture was grown for an additional 8 hours at 30° C. Cells were harvested and then resuspended in a lysis buffer (20 mM Tris, 300 mM NaCl, 5 mM 2-mercaptoethanol, 10% glycerol, 6M guanidinium-HCl), 1 mM PMSF and 0.5 ug/ml pepstatin. The cells were sonicated on ice for 30 seconds. The cell lysates were isolated after centrifugation at 8,000 RPM for 10 minutes. The cell lysates were then analyzed on Bio-Rad 16.5% Tris-Tricine Ready Gel. Recombined plasmids from the clones that could overexpress desired proteins were sequenced again to insure no unwanted recombination.

Protein Expression

The clones that could overexpress desired proteins were grown in 50 ml LB medium containing 100 ug/ml ampicillin for 16 hours at 37° C.; 10 ml of this overnight culture was added to 500 ml of LB medium with 100 ug/ml ampicillin and grown at 30° C. until late-log phase (˜15 hours). IPTG was added to a final concentration of 1 mM, and the culture was grown for an additional 8 hours at 30° C.

Protein Purification and Characterization

Protocols for protein purification have been described in detail elsewhere (Lajmi, A. R., et al., Prot. Exp. Purif., 2000, 18, 394-403). A brief summary of these procedures follows: after protein expression, cells were harvested and then resuspended in a lysis buffer. The cells were lysed by sonication. Sonicated cells were centrifuged. From the resulting supernatant, the 6×His-tagged proteins were isolated with TALON cobalt metal-ion affinity resin. The proteins were further purified by HPLC on a C4 column. The protein purification was monitored with SDS-PAGE gels. The characterization and the purities of the proteins were assessed on ESI-MS. Purified proteins were stored in guanidine protein storage buffer (2 M guanidine HCl, 50 mM NaCl, 10 mM phosphate, 5 mM EDTA, 5% glycerol, pH 7.4) with 1 mM PMSF and 1 ug/ml pepstatin at −80° C. for long-term storage or at −20° C. for short-term storage.

Temperature Leap of Renaturation of Proteins

Protein stocks were stored at −20° C. The amount of protein stock needed for the next experiments were renatured by the temperature-leap tactic developed by Xie and Wetlauer (Xie, Y. and Wetlaufer, D. B., Prot. Sci., 1996, 5, 517-523). The sequential dilutions were performed by adding proteins to the required amount of TKMC buffer (20 mM Tris, pH7.5, 4 mM KCl, 2 mM MgCl₂, 1 mM CaCl₂, 0.5 mM EDTA, 5% glycerol) at 4° C., incubating the diluted protein at 4° C.>2 hours, followed by rapid heating to 37° C. for 1 hour. The proteins were then immediately used for the next protein dilutions or EMSA experiments.

Radiolabeling of DNA Fragments

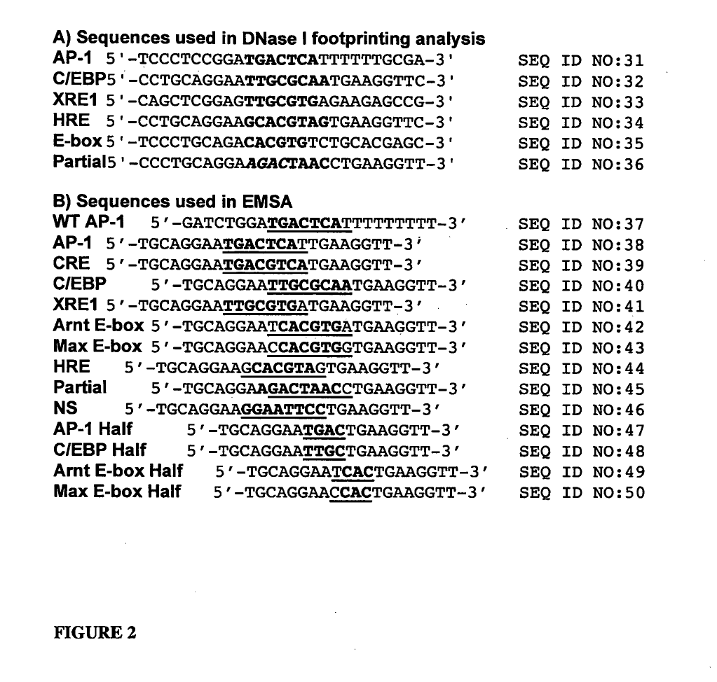

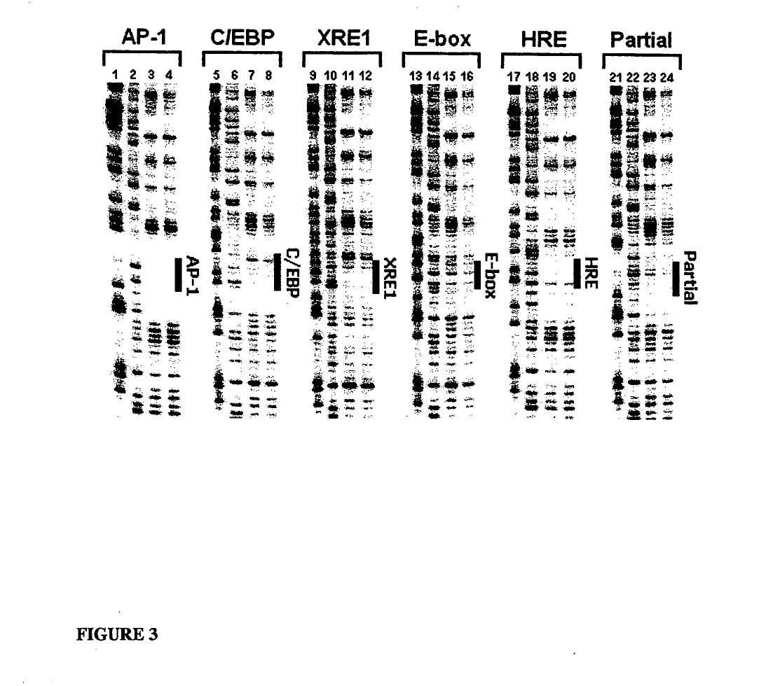

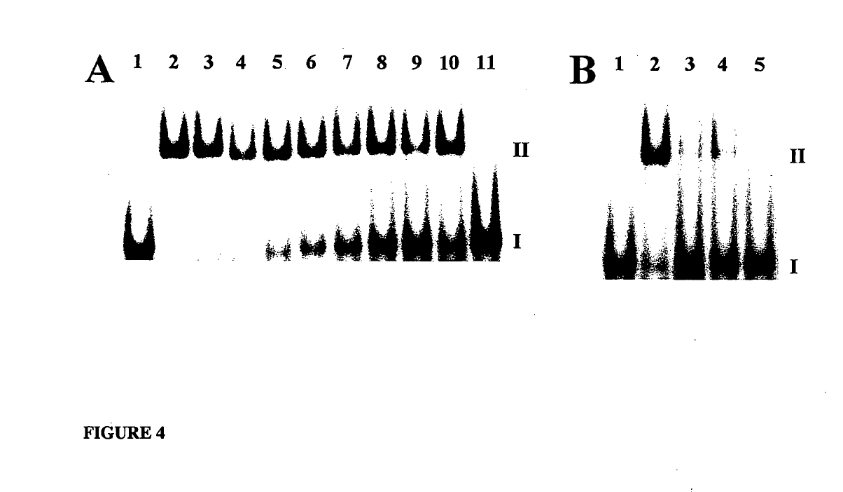

DNA oligonucleotides for DNAse I footprinting (FIG. 3) are shown in FIG. 2 and DNA oligonucleotides for EMSA experiments (FIG. 4) are shown in FIG. 2. DNA oligonucleotides used for EMSA experiments shown in FIG. 5 are listed below.

| | |
|---|---|
| WT AP-1 | |
| 5′-GATCTGGATGACTCATTTTTTTTT-3′ | SEQ ID NO:12 |
| 3′-CTAGACCTACTGAGTAAAAAAAAA-5′ | SEQ ID NO:13 |
| AP-1 | |
| 5′-TGCAGGAATGACTCATTGAAGGTT-3′ | SEQ ID NO:14 |
| 3′-ACGTCCTTACTGAGTAACTTCCAA-5′ | SEQ ID NO:15 |
| Max Ebox | |
| 5′-TGCAGGAACCACGTGGTGAAGGTT-3′ | SEQ ID NO:16 |
| 3′-ACGTCCTTGGTGCACCACTTCCAA-5′ | SEQ ID NO:17 |
| Arnt EBox | |
| 5′-TGCAGGAATCACGTGATGAAGGTT-3′ | SEQ ID NO:18 |
| 3′-ACGTCCTTAGTGCACTACTTCCAA-5′ | SEQ ID NO:19 |
| XRE1 | |
| 5′-TGCAGGAATTGCGTGATGAAGGTT-3′ | SEQ ID NO:20 |
| 3′-ACGTCCTTAACGCACTACTTCCAA-5′ | SEQ ID NO:21 |
| CEBP | |
| 5′-TGCAGGAATTGCGCAATGAAGGTT-3′ | SEQ ID NO:22 |
| 3′-ACGTCCTTAACGCGTTACTTCCAA-5′ | SEQ ID NO:23 |
| HRE | |
| 5′-TGCAGGAAGCACGTAGTGAAGGTT-3′ | SEQ ID NO:24 |
| 3′-ACGTCCTTCGTGCATCACTTCCAA-5′ | SEQ ID NO:25 |
| NON1 | |
| 5′-TGCAGGAAGGAATTCCTGAAGGTT-3′ | SEQ ID NO:26 |
| 3′-ACGTCCTTCCTTAAGGACTTCCAA-5′ | SEQ ID NO:27 |
| NON2 | |
| 5′-TGCAGGAAGACTAACCTGAAGGTT-3′ | SEQ ID NO:28 |
| 3′-ACGTCCTTCTGATTGGACTTCCAA-5′ | SEQ ID NO:29 |

Annealed oligonucleotides were 3′-end-double-labeled with [α-³²P]-dTTP and DNA polymerase I, Klenow fragment. The labeled DNA fragment was purified by ethanol precipitation and further purified by mini Quick Spin DNA Columns (Roche).

Electrophoretic Mobility Shift Assay (EMSA) (FIG. 5 experiment)

Reaction mixtures are 20 microliters total volume, containing [α-³²P]-dTTP-endlabeled DNA fragments (˜3000 cpm per reaction mixture), renatured proteins, TKMC buffer, 100 ug/ml acetylated BSA (sigma), 2 ug/ml poly(dl-dC) and 10% glycerol. Reactions were incubated at 4° C.>2 hours, then at 37° C. for 1 hour, followed by incubating at room temperature for another 30 minutes. 18 microliters of reaction mixture was then directly loaded into a 10% acrylamide, 1:29 crosslinked, non-denaturing PAGE gel. Gels were loaded while running at 110 V. Gels were run for 100 minutes at 110 V. The gels were dried and exposed to phosphor screens for ˜20 hours.

The radioactive signals were visualized and quantified by a Phosphorlmager (Storm 840; Molecular Dynamics, Amersham Biosciences) and ImageQuant ver. 5.2 software (Molecular Dynamics). The EMSA data were fit to the Langmuir equation by using Kaleidagraph ver. 3.6. Quantitative EMSA data were analyzed as described earlier (Bird, G. H. et al., Biopolymers, 2002, 65, 10-20).

Electrophoretic Gel Mobility Shift Assay (EMSA). (FIG. 3 Experiment)

24-mer duplexes were double-labeled at the 3′-terminus with [α-³²P]-dTTP. Each duplex contains a core target site surrounded by the same 3′ and 5′ flanking sequences, which were chosen to minimize DNA secondary structure. Solid wt bZIP was freshly dissolved in EMSA buffer (20 mM Tris and 1 mM phosphate, pH 7.5, 5 mM NaCl, 4 mM KCl, 2 mM MgCl₂, 1 mM CaCl₂, 1 mM EDTA, 100 μg/ml non-acetylated BSA, 2 μg/ml poly(dl-dC), 200 mM guanidine HCl, and 10% glycerol). Protein solutions were boiled at 90° C. for 10 minutes, and slowly cooled to room temperature over 4 hours. 3′-endlabeled DNA duplexes were then added to protein solutions on ice. Each reaction contained -3000 cpm labeled DNA duplexes in EMSA buffer. Reactions were incubated at 4° C. overnight, 37° C. for 1 hour, and room temperature for 30 min. Reactions were loaded onto a 10% polyacrylamide, 1:29 crosslinked, nondenaturing gel. Gels were loaded while running at 120 V and run for 3 hours. Gels were dried and autoradiographed as above.

Determination of K_{d} Values.

The volumes of the bands corresponding to free and bound DNA were quantified using Molecular Dynamics ImageQuant software (version 5.2). All gels were run under equilibrium binding conditions. The bound-DNA fractions vs. protein monomer concentrations were fit to Eq. (1), a modified two-state binding equation for determination of apparent dissociation constants, with KaleidaGraph software (version 3.6.4, Synergy Software).(Metallo and Schepartz 1994; Bird et al. 2002)
θₐₚₚ=θₘᵢₙ+(θₘₐₓ−θₘᵢₙ)[1/(1+K_{d}²/[M]²)] (1)

where K_{d} corresponds to the apparent monomeric dissociation constant, [M] is the concentration of monomeric wt bZIP, θₘᵢₙ is the bound-DNA fraction when no wt bZIP present, and θₘₐₓ is the bound-DNA fraction when DNA binding is saturated. Only data sets fit to Eq. (1) with R values>0.970 are reported in Table I. Each dissociation constant was determined from the average of two independent data sets.

TABLE 1
Dissociation Constants for Synthetic and
Expressed wt bZIP bound to DNA Sites

K_{d} (10⁻⁹ M)^{a}

| Binding Site | Synthetic wt bZIP | Expressed wt bZIP^{b} |
|---|---|---|
| WT AP-1 | 13 ± 0.38 | |
| AP-1 | 13 ± 0.59 | 4.8 ± 0.12^{b} |
| CRE | 12 ± 0.28 | |
| C/EBP | 120 ± 16 | 29 ± 3.6^{b} |
| XRE1 | 240 ± 18 | |
| Arnt E-box | 570 ± 0.69^{c} | |
| Max E-box | 840 ± 26^{c} | |
| HRE | 1400 ± 160^{c} | |
| Partial | 280 ± 49 | |
| AP-1 Half | 84 ± 0.01 | 23 ± 8.7^{b} |
| C/EBP Half | >5000^{c} | >1500^{c} |
| Arnt Ebox Half | >4000^{c} | |
| Max Ebox Half | no activity^{d} | |
| NS | no activity^{d} | no activity^{d} |

^{a}Average values of dissociation constants were obtained from two independent experiments, R values >0.97.
^{b}Higher-order bandshifts were not included in calculation of dissociation constants (see text and Supplemental Data).
^{c}Saturation protein binding was not achieved in these titrations.
^{d}The concentration of wt bZIP ranged from 0-1000 nM.

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

### REFERENCES

Amati B, Land H (1994) Myc-Max-Mad: a transcription factor network controlling cell cycle progression, differentiation and death. Curr Opin Gene Dev 4(102-108).
Amati B, Brooks M W, Levy N, Littlewood T D, Evan G I et al. (1993) Oncogenic Activity of the c-Myc Protein Requires Dimerization with Max. Cell 72: 233-245.
Bertazzi P A, Pesatori A C, Consonni D, Tironi A, Landi M T et al. (1993) Cancer Incidence in a Popuylation Accidentally Exposed to Dioxin. Epidemiology 4: 398-406.
Bird G H, Lajmi A R, Shin J A (2002) Sequence-Specific Recognition of DNA by Hydrophobic, Alanine-scanning Mutants of the bZIP Motif Investigated by Fluorescence Anisotropy. Biopolymers 65: 10-20.
Blackwell T K, Huang J, Ma A, Kretzner L, Alt F W et al. (1993) Binding of myc proteins to canonical and noncanonical DNA sequences. Mol Cell Biol 13: 5216-5224.
Blackwood E M, Eisenman R N (1991) Max: A Helix-Loop-Helix Zipper Protein That Forms a Sequence-Specific DNA-Binding Complex with Myc. Science 251: 1211-1217.
Brownlie P, Ceska T A, Lamers M, Romier C, Stier G et al. (1997) The crystal structure of an intact human Max-DNA complex: new insights into mechanisms of transcriptional control. Structure 5: 509-520.
Cadepond F, Schweizer-Groyer G, Segard-Maurel I, Jibard N, Hollenberg S M et al. (1991) Heat Shock Protein 90 as a Critical Factor in Maintaining Glucocorticosteroid Receptor in a Nonfunctional State. J Biol Chem 266: 5834-5841.
Crosby D G, Wong A S, Plimmer J R, Woolson E A (1971) Photodecomposition of Chlorinated Dibenzo-p-Dioxins. Science 173: 748-749.
Dalla-Favera R, Bregni M, Erikson J, Patterson D, Gallo R C et al. (1982) Human c-myc oncogene is located on the region of chromosome 8 that is translocated in Burkift lymphoma cells. Proc Natl Acad Sci USA 79: 7824-7827.
Dawson P E, Kent S B H (1993) Convenient total synthesis of a four-helix TASP molecule by chemoselective ligation. J Am Chem Soc 115: 7263-7266.
Ellenberger T, Fass D, Arnaud M, Harrison S C (1994) Crystal structure of transcription factor E47: E-box recognition by a basic region helix-loop-helix. dimer. Genes Dev 8: 970-980.
Ellenberger T E, Brandl C J, Struhl K, Harrison S C (1992) The GCN4 basic region leucine zipper binds DNA as a dimer of uninterrupted a helices: Crystal stucture of the protein-DNA complex. Cell 71: 1223-1237.
Ferre-D'Amare A R, Prendergast G C, Ziff E B, Burley S K (1993) Recognition by Max of its cognate DNA through a dimeric b/HLH/Z domain. Nature 363: 3845.
Ferre-D'Amare A R, Pogonec P, Roeder R G, Burley S K (1994) Structure and function of the b/HLH/Z domain of USF. EMBO J 13:180-189.
Fingerhut M A, Halperin W E, Marlow D A, Piagitelli L A, Honchar P A et al. (1991) Cancer Mortality in Workers Exposed to 2,3,7,8-Tetrachlorodibenzo-p-dioxin. N Engl J Med 324: 212-218.
Fisher J M, Jones K W, Whitlock J P (1989) Activation of Transcription as a General Mechanism of 2,3,7,8-Tetrachlorodibenzo-p-Dioxin Action. Mol Carcinogen 1: 216-221.
Fujisawa-Sehara A, Sogawa K, Yamane M, Fujii-Kuriyama Y (1987) Characterization of xenobiotic responsive elements upstream from the drug-metabolizing cytochrome P-450c gene: a similarity to glucocorticoid regulatory elements. Nucl Acids Res 15: 4179-4191.
Gardner L, Lee L, Dang C (2002) The c-Myc Oncogenic Transcription Factor. In: Bertino J R, editor. Encyclopedia of Cancer. San Diego, Calif.: Academic Press.
Ghadiri M R, Soares C, Choi C (1992) Design of an artificial four-helix bundle metalloprotein via a novel ruthenium (II)-assisted self-assembly process. J Am Chem Soc 114: 4000-4002.
Glover J N M, Harrison S C (1995) Crystal structure of the heterodimeric bZIP transcription factor c-Fos-c-Jun bound to DNA. Nature 373: 257-261.
Gradin K, McGuire J, Wenger R H, Kvietikova I, Whitelaw M L et al. (1996) Functional Interference between Hypoxia and Dioxin Signal Transduction Pathways: Competition for Recruitment of the Arnt Transcription Factor. Mol Cell Biol 16: 5221-5231.
Gray L E J, Ostby J S (1995) In utero 2,3,7,8tetrachlorodibenzo-p-dioxin (TCDD) alters reproductive morphology and function in female rat offspring. Toxicol Appl Pharmacol 133: 285-294.
Grove A, Mutter M, Rivier J E, Montal M (1993) Template-assembled synthetic proteins designed to adopt a globular, four-helix bundle conformation form ionic channels in lipid bilayers. J Am Chem Soc 115: 5919-5924.
Hogenesch J B, Chan W K, Jackiw V H, Brown R C, Gu Y-Z et al. (1997) Characterization of a Subset of the Basic-Helix-Loop-Helix-PAS Superfamily That Interacts with Components of the Dioxin Signaling Pathway. J Biol Chem 272: 8581-8593.
Hollenbeck J J, Oakley M G (2000) GCN4 Binds with High Affinity to DNA Sequences Containing a Single Consensus Half-Site. Biochemistry 39: 6380-6389.
Jakoby M, Weisshaar B, Droge-Laser w, Vicente-Carbajosa J, Tiedemann J et al. (2002) bZIP transcription factors in Arabidopsis. Trends Plant Sci 7: 106-111.
Johnson E F (1991) A Partnership between the Dioxin Receptor and a Basic Helix-Loop-Helix Protein. Science 252: 924.

Johnson P F (1993) Identification of C/EBP basic region residues involved in DNA sequence recognition and half-site spacing preference. Mol Cell Biol 13: 6919-6930.

Keller W, König P, Richmond T J (1995) Crystal structure of a bZIP/DNA Complex at 2.2 Å: Determinants of DNA specific recognition. J Mol Biol 254: 657-667.
König P, Richmond T J (1993) The X-ray structure of the GCN4-bZIP bound to ATF/CREB site DNA shows the complex depends on DNA flexibility. J Mol Biol 233:139-154.
Lajmi A R, Wallace T R, Shin J A (2000a) Short, Hydrophobic, Alanine-based Proteins Based on the bZIP Motif: Overcoming Inclusion Body Formation and Protein Aggregation During Overexpression, Purification, and Renaturation. Prot Exp Purif 18: 394-403.
Lajmi A R, Lovrencic M E, Wallace T R, Thomlinson R R, Shin J A (2000b) Minimalist, Alanine-based, Helical Protein Dimers Bind to Specific DNA Sites. J Am Chem Soc 122: 5638-5639.
Lando D, Gorman J J, Whitelaw M L, Peet D J (2003) Oxygen-dependent regulation of hypoxia-inducible factors by prolyl and asparaginyl hydroxylation. Eur J Biochem 270: 781-790.
Landschulz W H, Johnson P F, McKnight S L (1988) The Leucine Zipper: A Hypothetical Structure Common to a New Class of DNA Binding Proteins. Science 240: 1759-1764.
Luque I, Mayorga O L, Freire E (1996) Structure-based thermodynamic scale of α-helix propensities in amino acids. Biochemistry 35: 13681-13688.
Ma P C M, Rould M A, Weintraub H, Pabo C O (1994) Crystal Structure of MyoD bHLH Domain-DNA Complex: Perspectives on DNA Recognition and Implications for Transcriptional Activation. Cell 77: 451-459.
Marqusee S, Baldwin R L (1987) Helix stabilization by Glu-Lys salt bridges in short peptides of de novo design. Proc Natl Acad Sci USA 84: 8898-8902.
Metallo S J, Schepartz A (1994) Distribution of labor among bZIP segments in the control of DNA affinity and specificity. Chem Biol 1: 143-151.
Nair S K, Burley S K (2003) X-Ray Structure of Myc-Max and Mad-Max Recognizing DNA: Molecular Bases of Regulation by Proto-Oncogenic Transcription Factors. Cell 112: 193-205.
Nau M M, Brooks B M, Battey J, Sausville E, Gazdar A F et al. (1985) L-Myc, a new Myc-related gene amplified and expressed in human small cell lung cancer. Nature 318: 69-73.
Nesbit C D, Tersak J M, Prochownik E V (1999) MYC oncogenes and human neoplastic disease. Oncogene 18: 3004-3016.
O'Hagan R C, Schreiber-Agus N, Chen K, David G, Engelman J A et al. (2000) Gene-target recognition among members of the Myc superfamily and implications for oncogenesis. Nat Genet 24: 113-119.
O'Neil K T, DeGrado W F (1990) A thermodynamic scale for the helix-forming tendencies of the commonly occurring amino acids. Science 250: 646-651.
O'Neil K T, Shuman J D, Ampe C, DeGrado W F (1991) DNA-induced increase in the α-helical content of C/EBP and GCN4. Biochemistry 30: 9030-9034.
Orian A, van Steensel B, Delrow J, Bussemaker H J, Li L et al. (2003) Genomic binding by the Drosophila Myc, Max, Mad/Mnt transcription factor network. Genes Dev 17:1101-1114.
Poland A, Knutson J C (1982) 2,3,7,8-Tetrachlorodibenzopdioxin and related halogenated aromatic hydrocarbons: examination of the mechanism of toxicity. Ann Rev Pharmacol Toxicol 22: 517.
Pollenz R S, Sattler C A, Poland A (1995) The Aryl Hydrocarbon Receptor and Aryl Hydrocarbon Receptor Nuclear Translocator Protein Show Distinct Subcellular Localization in Hepa 1c1c7 Cells by Immunofluorescence Microscopy. Mol Pharmacol 45: 428-438.
Pongratz I, Antonsson C, Whitelaw M L, Poellinger L (1998) Role of the PAS Domain in Regulation of Dimerization and DNA Binding Specificity of the Dioxin Receptor. Mol Cell Biol 18: 4079-4088.
Reisman D, Elkind N B, Roy B, Beamon J, Rofter V (1993) c-Myc Transactivates the p53 Promoter through a Required Downstream CACGTG Motif. Cell Growth Differ 4: 57-65.
Reyes H, Reisz-Porszasz S, Hankinson O (1992) Identification of the Ah Receptor Nuclear Translocator Protein (Arnt) as a Component of the DNA Binding Form of the Ah Receptor. Science 256: 1193-1195.
Roberts L (1991) Research News: Dioxin Risks Revisited. Science 251: 624-626.
Rowlands C, Krishnan V, Wang X, Santostefano M, Safe S et al. (1993) Characterization of the Aryl hydrocarbon Receptor and Aryl Hydrocarbon Responsiveness in Human Ovarian Carcinoma Cell Lines. Cancer Res 53: 1802-1807.
Sasaki T, Kaiser E (1989) Helichrome: Synthesis and Enzymatic Activity of a Designed Hemiprotein. J Am Chem Soc 111(380-381).
Saudek V, Pasley H S, Gibson T, Gausepohl H, Frank R et al. (1991) Solution structure of the basic region from the transcriptional activator GCN4. Biochemistry 30: 1310-1317.
Schwab M, Varmus H E, Bishop J M, Grzeschik K H, Naylor S L et al. (1984) Chromosome localization in normal human cells and neuroblastomas of a gene related to c-Myc. Nature 308: 288-291.
Shin J A (1997) Specific DNA binding peptide derivatized solid support. Bioorg Med Chem Left 7: 2367-2372.
Struhl K (1989) Helix-turn-helix, zinc-finger, and leucine-zipper motifs for eucaryotic transcriptional regulatory proteins. Trends Biochem Sci 14: 137-140.
Taub R, Kirsch I, Morton C, Lenoir G, Swan D et al. (1982) Translocation of the c-myc gene into the immunoglobulin heavy chain locus in human Burkift lymphoma and murine plasmacytoma cells. Proc Natl Acad Sci USA 79: 7837-7841.
Vinson C R, Sigler P B, McKnight S L (1989) Scissors-Grip Model for DNA Recognition by a Family of Leucine Zipper Proteins. Science 246: 911-916.
Weiss M A, Ellenberger T E, Wobbe C R, Lee J P, Harrison S C et al. (1990) Folding transition in the DNA-binding domain of GCN4 on specific binding to DNA. Nature 347: 575-578.
Wharton R P, Ptashne M (1985) Changing the Binding Specificity of a Repressor by Redesigning an α-helix. Nature 316: 601-605.
Whitelaw M, Pongratz I, Wilhelmsson A, Gustafsson J-A, Poellinger L (1993) Ligand-Dependent Recruitment of the Arnt Coregulator Determines DNA Recognition of the Dioxin Receptor. Mol Cell Biol 13: 2504-2514.
Wu L, Whitlock J P (1993) Mechanism of dioxin action: receptor-enhancer interactions in intact cells. Nuci Acid Res 21: 119-125.
Xie Y, Wetlaufer D B (1996) Control of aggregation in protein refolding: The temperature-leap tactic. Prot Sci 5: 517-523.
Zervos A S, Gyuris J, Brent R (1993) Mxi1, a Protein hat Specifically Interacts with Max to Bind Myc-Max Recognition Sites. Cell 72: 223-232.

1. A method of specifically targeting a noncanonical gene regulatory sequence comprising administering to a cell or animal in need thereof a minimalist basic region-leucine zipper bZIP derivative, wherein the minimalist basic region-leucine zipper bZIP derivative binds the noncanonical gene regulatory sequence.

2. The method of claim 1, wherein the basic region and/or leucine zipper of the basic region-leucine zipper bZIP derivative is derived from a bZIP protein selected from the group consisting of Fos, Jun, GCN4 (general control of nitrogen and purine metabolism factor-4, VBP (vitellogenin gene binding protein), GBF-1 (G-box factor-1), opaque, DBP (D-box binding protein), CHOP-10 (C/EBP homologous protein-I0), CREB (CRE binding protein), C/EBP (CCAAT/enhancer binding protein), PAR (proline- and acidic amino acid-rich protein) and ATF2 (activating transcription factor-2).

3. The method of claim 1, wherein the basic region of the basic region-leucine zipper bZIP derivative is derived from GCN4 and the leucine zipper region of the basic region-leucine zipper bZIP derivative is derived from C/EBP.

4. The method of claim 1, wherein the noncanonical gene regulatory sequence is selected from the group consisting of C/EBP (CCAAT/enhancer binding protein, 5′-TTGCGCAA), XRE1 (xenobiotic response element, 5′-TTGCGTGA), HRE (HIF response element, 5′-GCACGTAG), and two E-box derivatives (5′-TCACGTGA, preferred site for Max and Myc; 5′-CCACGTGG, preferred site of Arnt).

5. The method of claim 1, wherein the noncanonical sequence is normally recognized by Max.

6. The method of claim 1, wherein the noncanonical sequence is normally recognized by Myc.

7. The method of claim 1, wherein the noncanonical sequence is normally recognized by Arnt.

8. The method of claim 1, wherein the noncanonical sequence is normally recognized by HIF.

9. The method of claim 1, wherein the basic region of the basic region-leucine zipper bZIP derivative has alanine substitutions.

10. The method of claim 9, wherein the basic region is derived from GCN4.

11. The method of claim 10, wherein the basic region has 4, 11 or 18 alanine substitutions.

12. The method of claim 6 for repressing myc-related diseases.

13. The method of claim 6 for treating cancer.

14. The method of claim 13, wherein the cancer is selected from the group consisting of Burkitt lymphoma, neuroblastoma small cell lung cancer, breast cancer, colon cancer, liver cancer and gynecological cancer.

15. The method of claim 13, wherein the minimalist bZIP derivative is fused to a repressor.

16. The method of claim 8 for repressing HIF-related diseases.

17. The method of claim 16, wherein the minimalist bZIP derivative is fused to a repressor.

18. The method of claim 7 for modulating the dioxin pathway.

19. The method of claim 18, wherein the basic region-leucine zipper minimalist bZIP derivative is fused to a repressor.

20. The method of claim 19 for repressing the dioxin pathway.

21. The method of claim 18, wherein the basic region-leucine zipper minimalist bZIP derivative is fused to an activator.

22. The method of claim 21 for activating the dioxin pathway.

23. The method of claim 18, for treating cancer.

24. The method of claim 23, wherein the cancer is selected from the group consisting of liver cancer, cancer of the lymph nodes, multiple myeloma, leukemia, soft tissue sarcoma, non-Hodgkin's lymphoma and respiratory cancer.

25. The method of claim 1 for modulating G-box regulated genes in a plant cell.


SEQUENCE LISTING (50 sequences)

SEQ ID NO: 1 - DNA, 7 bp, Artificial sequence
  AP-1 binding site
tgactca

SEQ ID NO: 2 - protein, 27 aa, Artificial sequence
  basic region wild type bZIP peptide
DPAALKRARNTEAARRSRARKLQRMKQ

SEQ ID NO: 3 - protein, 27 aa, Artificial sequence
  basic region 4A bZIP mutant peptide
ARAAAARARNTAAARRSRARKLQRMKQ

SEQ ID NO: 4 - protein, 27 aa, Artificial sequence
  basic region 11A bZIP mutant peptide
ARAAAARARNTAAARRSRAAKAAAAAA

SEQ ID NO: 5 - protein, 27 aa, Artificial sequence
  basic region 18A bZIP mutant peptide
AAAAAAAAANAAAAAAARAAKAAAAAA

SEQ ID NO: 6 - DNA, 8 bp, Artificial sequence
  CRE binding site
tgacgtca

SEQ ID NO: 7 - DNA, 8 bp, Artificial sequence
  C/EBP regulatory sequence
ttgcgcaa

SEQ ID NO: 8 - DNA, 8 bp, Artificial sequence
  XRE1 regulatory sequence
ttgcgtga

SEQ ID NO: 9 - DNA, 8 bp, Artificial sequence
  HRE regulatory sequence
gcacgtag

SEQ ID NO: 10 - DNA, 8 bp, Artificial sequence
  E-box preferred binding site for Max and Myc transcription factors
tcacgtga

SEQ ID NO: 11 - DNA, 8 bp, Artificial sequence
  E-box preferred binding site for Arnt transcription factor
ccacgtgg

SEQ ID NO: 12 - DNA, 24 bp, Artificial sequence
  oligonucleotide
gatctggatgactcattttttttt

SEQ ID NO: 13 - DNA, 24 bp, Artificial sequence
  oligonucleotide
aaaaaaaaatgagtcatccagatc

SEQ ID NO: 14 - DNA, 24 bp, Artificial sequence
  oligonucleotide
tgcaggaatgactcattgaaggtt

SEQ ID NO: 15 - DNA, 24 bp, Artificial sequence
  oligonucleotide
aaccttcaatgagtcattcctgca

SEQ ID NO: 16 - DNA, 24 bp, Artificial sequence
  oligonucleotide
tgcaggaaccacgtggtgaaggtt

SEQ ID NO: 17 - DNA, 24 bp, Artificial sequence
  oligonucleotide
aaccttcaccacgtggttcctgca

SEQ ID NO: 18 - DNA, 24 bp, Artificial sequence
  oligonucleotide
tgcaggaatcacgtgatgaaggtt

SEQ ID NO: 19 - DNA, 24 bp, Artificial sequence
  oligonucleotide
aaccttcatcacgtgattcctgca

SEQ ID NO: 20 - DNA, 24 bp, Artificial sequence
  oligonucleotide
tgcaggaattgcgtgatgaaggtt

SEQ ID NO: 21 - DNA, 24 bp, Artificial sequence
  oligonucleotide
aaccttcatcacgcaattcctgca

SEQ ID NO: 22 - DNA, 24 bp, Artificial sequence
  oligonucleotide
tgcaggaattgcgcaatgaaggtt

SEQ ID NO: 23 - DNA, 24 bp, Artificial sequence
  oligonucleotide
aaccttcattgcgcaattcctgca

SEQ ID NO: 24 - DNA, 24 bp, Artificial sequence
  oligonucleotide
tgcaggaagcacgtagtgaaggtt

SEQ ID NO: 25 - DNA, 24 bp, Artificial sequence
  oligonucleotide
aaccttcactacgtgcttcctgca

SEQ ID NO: 26 - DNA, 24 bp, Artificial sequence
  oligonucleotide
tgcaggaaggaattcctgaaggtt

SEQ ID NO: 27 - DNA, 24 bp, Artificial sequence
  oligonucleotide
aaccttcaggaattccttcctgca

SEQ ID NO: 28 - DNA, 24 bp, Artificial sequence
  oligonucleotide
tgcaggaagactaacctgaaggtt

SEQ ID NO: 29 - DNA, 24 bp, Artificial sequence
  oligonucleotide
aaccttcaggttagtcttcctgca

SEQ ID NO: 30 - protein, 65 aa, Artificial sequence
  GCN4 basic region C/EBP leucine zipper linker
DPAALKRARNTEAARRSRARKLQRMKQLEQKVLELTSDNDRLRKRVEQLSRELDTLGGCGGYYYY

SEQ ID NO: 31 - DNA, 27 bp, Artificial sequence
  oligonucleotide
tccctccggatgactcattttttgcga

SEQ ID NO: 32 - DNA, 27 bp, Artificial sequence
  oligonucleotide
cctgcaggaattgcgcaatgaaggttc

SEQ ID NO: 33 - DNA, 27 bp, Artificial sequence
  oligonucleotide
cagctcggagttgcgtgagaagagccg

SEQ ID NO: 34 - DNA, 27 bp, Artificial sequence
  oligonucleotide
cctgcaggaagcacgtagtgaaggttc

SEQ ID NO: 35 - DNA, 27 bp, Artificial sequence
  oligonucleotide
tccctgcagacacgtgtctgcacgagc

SEQ ID NO: 36 - DNA, 27 bp, Artificial sequence
  oligonucleotide
ccctgcaggaagactaacctgaaggtt

SEQ ID NO: 37 - DNA, 24 bp, Artificial sequence
  oligonucleotide
gatctggatgactcattttttttt

SEQ ID NO: 38 - DNA, 24 bp, Artificial sequence
  oligonucleotide
tgcaggaatgactcattgaaggtt

SEQ ID NO: 39 - DNA, 24 bp, Artificial sequence
  oligonucleotide
tgcaggaatgacgtcatgaaggtt

SEQ ID NO: 40 - DNA, 24 bp, Artificial sequence
  oligonucleotide
tgcaggaattgcgcaatgaaggtt

SEQ ID NO: 41 - DNA, 24 bp, Artificial sequence
  oligonucleotide
tgcaggaattgcgtgatgaaggtt

SEQ ID NO: 42 - DNA, 24 bp, Artificial sequence
  oligonucleotide
tgcaggaatcacgtgatgaaggtt

SEQ ID NO: 43 - DNA, 24 bp, Artificial sequence
  oligonucleotide
tgcaggaaccacgtggtgaaggtt

SEQ ID NO: 44 - DNA, 24 bp, Artificial sequence
  oligonucleotide
tgcaggaagcacgtagtgaaggtt

SEQ ID NO: 45 - DNA, 24 bp, Artificial sequence
  oligonucleotide
tgcaggaagactaacctgaaggtt

SEQ ID NO: 46 - DNA, 24 bp, Artificial sequence
  oligonucleotide
tgcaggaaggaattcctgaaggtt

SEQ ID NO: 47 - DNA, 20 bp, Artificial sequence
  oligonucleotide
tgcaggaatgactgaaggtt

SEQ ID NO: 48 - DNA, 20 bp, Artificial sequence
  oligonucleotide
tgcaggaattgctgaaggtt

SEQ ID NO: 49 - DNA, 20 bp, Artificial sequence
  oligonucleotide
tgcaggaatcactgaaggtt

SEQ ID NO: 50 - DNA, 20 bp, Artificial sequence
  oligonucleotide
tgcaggaaccactgaaggtt